(19) United States
(12) Patent Application Publication
(10) Pub. No.: US 2022/0296083 A1
(43) Pub. Date: Sep. 22, 2022

(54) ARTHROSCOPY METHOD AND DEVICE

(71) Applicants: Innovation Advances Ltd, Bradford Avon (GB); Trice Medical, Inc., Malvern, PA (US)

(72) Inventors: Adrian Wilson, Bradford Avon (GB); Kristian Saksida, Bradford Avon (GB); Kristian Kley, Bradford Avon (GB); Mark D. Foster, Malvern, PA (US); Sarah E. Sachinis, Malvern, PA (US); Alfred J. Intintoli, West Chester, PA (US)

(21) Appl. No.: 17/596,818

(22) PCT Filed: Jun. 19, 2020
(86) PCT No.: PCT/US2020/038812

§ 371 Date: Dec. 17, 2021

(30) Foreign Application Priority Data
Jun. 21, 2019 (GB) 1908924.2

(51) Int. Cl. A61B 1/00; A61B 1/317; A61B 17/17; A61B 17/16; A61B 1/04

(52) U.S. Cl. CPC A61B 1/00096; A61B 1/317; A61B 17/1764; A61B 1/00105; A61B 17/1675; A61B 1/00165; A61B 1/00154; A61B 1/042; A61B 2034/301

(57) ABSTRACT

Methods and devices for obtaining an image of a target area of a joint in a subject are disclosed. Such methods and devices may involve positioning at least a part of a device in an aperture in a bone involved in the joint such that the image capture component is in operable relation to the target area. The methods and devices disclosed herein may also be used for and in combination with ligament repair. Methods of surgery utilising this method and devices suitable for use with this method are also disclosed.

### TECHNICAL FIELD

The present disclosure relates to methods and systems for visualising a target area of a joint during arthroscopic joint inspection and joint surgery.

### BACKGROUND

Arthroscopy is a technique used to visualise and/or carry out “key hole” surgery inside any joint, in order to provide diagnosis and/or treatment. The technique can be used, for example, to inspect the state of the joint cartilage, remove torn joint cartilage, and inspect and repair torn joint tissue such as the joint surface cartilage, ligaments and, in the knee, the meniscal cartilages.

The technique may be carried out with an arthroscope (often referred to more simply as a “scope”), which is an instrument usually comprising a hollow tube carrying a light source and lens. The arthroscope can be used to capture images from inside the joint and relay these outside the body, where they can be displayed on a screen. The diameter of the arthroscope tube varies depending on the size of the joint. A tube of 3.5-4.5 mm in diameter may be utilised with the knee joint. An arthroscope for a large joint such as the knee, hip or shoulder may be 3.5 mm Smaller scopes exist for use in smaller joints. The tip of the arthroscope may be angled so that the different areas of the joint space can be seen simply by rotating the scope. An angle of 30° is may be used. However, arthroscopes having angles such as 45° and 70° and 90° can be used.

The scope may be introduced into the joint via a sheath (35), shown in FIG. 9A. This not only accommodates the scope but may also have one or two attachments that allow inflow (37) and outflow (38) of fluid to dilate and wash the joint, in order to aid visualisation.

The arthroscope is inserted into an incision made near the joint, through a channel made in the soft tissue and into the joint. One or more further incisions at locations around the joint can be utilised to provide other access points into the joint, to achieve different viewpoints or to provide an access for surgical instruments. These access routes/channels may be arthroscopic portals.

An arthroscopic surgical procedure may be carried out with one working portal and one portal for visualisation, with these two portals being interchanged during the procedure. However, additional portals around the joint to be used as well, to try to obtain a better view of a particular area of the joint. Standard portals with safe access to the joint, and which offer specific views inside a joint, have been defined for each joint. Portals for the knee joint are shown schematically in FIG. 1. The first portal may be established blind and the scope may then beintroduced to the first portal. Then the second portal may be made under direct visualisation using the scope.

Initially the sheath with its inner obturator are introduced and then the obturator is removed and replaced with the scope which is inserted into the sheath. As shown in FIG. 9B the obturator (39) may include a blunt end (40) and a handle (41).

Surgery performed using arthroscopy is known as keyhole surgery or minimally invasive surgery as it limits the size of the incision needed and therefore reduces pain and healing time, as well as lowering the risk of infection.

However, medical procedures involving the use of arthroscopes may suffer from certain limitations. The medical professional is often not able to look directly at the area of interest within the joint as the position of the arthroscope is constrained by the anatomy of the joint, especially the bony anatomy of the joint. The surgeon is also limited by the important structures surrounding the joint, such as the nerves and blood vessels. For example, in the knee a surgeon may look and work from the front of the knee by making stab incisions either side of the patella tendon. One incision may be made on each side of the patella tendon, which allow the surgeon to get somewhat limited views of the knee joint. For instance, it is difficult to see the front of the knee as the telescope does not look back on itself and is introduced from the front. The surgeon can make additional portals but these still provide a limited view.

As such, a less than ideal viewpoint must be used to conduct the joint inspection or surgery. Where surgery is being performed using two or more different portals to provide a view of the area of interest the medical professional also has to deal with problems of parallax. Moreover, the seal between the scope and the soft tissues is not water tight, so insufflated fluid introduced into the joint can track back along the incision track made into the joint. Further, moving the scope from one portal to another also allows fluid to escape. Escaping fluid causes inevitable soft tissue swelling and this can make the surgery difficult. This is especially true in the shoulder.

The use of arthroscopes is therefore a skill that takes a significant amount of time to acquire. In particular, during surgery the surgeon can be required to hold the arthroscope in one hand while the other hand introduces medical instruments into another portal and performs the surgical procedure. Alternatively, should the surgeon require two hands to perform the medical procedure a surgical assistant is needed to hold the arthroscope, requiring good teamwork and a skilled surgical assistant as well as a skilled surgeon. Junior surgeons and junior surgical assistants lacking sufficient practice and skill may struggle to maintain a constant view of the target area using the arthroscope, and the surgeon may intermittently have to stop the work that he or she is carrying out such that the scope can be placed once more into the correct position to provide a view of the target area.

Moreover, to get as good a view as possible it can be necessary to hold the arthroscope only 1 or 2 mm away from areas in the joint that, if damaged by the arthroscope, cause further problems. For example, in the knee joint there is a fat pad, which is a triangular structure at the front of the knee just below the knee cap and behind the patella tendon that connects the patella to the shin bone. The fat pad sits immediately behind an area of the joint where the arthroscope often needs to be positioned. If the arthroscope enters the fat pad by mistake, even for a few seconds, the fat pad rapidly expands with fluid and then significantly obstructs the view of the target area, making the procedure more difficult. This is a particular problem when the arthroscope is inserted through either the anterolateral or anteromedial portals, where if the arthroscope is withdrawn by just a few millimetres it may come into contact with the fat pad.

Still further, it is frequently necessary to move the joint during the medical procedure in order to obtain a good view of a particular target area. The arthroscope needs to be kept on target whilst the joint is being moved, and the surgeon or assistant needs to follow the movement and try to keep the view constant. It is also important that the arthroscope does not drift into areas such as back into the soft tissue portal through which the arthroscope has been placed, potentially causing further damage to these areas, or deeper into the joint where it can cause damage by scratching or scraping the delicate surfaces of the joint. In the knee, for instance, it can be necessary to place the joint into the so-called valgus position to obtain a view of the medial compartment. This requires the surgeon to stand between the outstretched leg and the operating table and then push the leg away from the table creating a strain that opens up the inner compartment of the knee. When this is complete the surgeon then positions the knee into a figure of four position which allows visualisation of the lateral compartment of the knee. In contrast, the patella femoral compartment can be visualized with the leg in full extension. During these joint manoeuvres the arthroscope must be retained in position.

As we move into the era of robotic surgery it has been noted that it will take some advanced technology to have the robot maintain position through soft tissue portals on a moving target.

A major cause of failure of arthroscopic surgery is inadequate visualisation of a target area within the joint. This is particularly the case in ligament reconstruction or repair surgery, where it is necessary to include fixation devices or drill holes into the ends of the bones involved in the joint. Inadequate visualization can mean that these are placed in less favourable positions leading to failure of the operation.

One such example is in anterior cruciate ligament (ACL) surgery. The ACL controls rotation within the knee and can tear as a result of an injury. Once it has torn the knee becomes unstable. The object of the arthroscopy surgery is either to replace the torn ACL with a graft. This is called an ACL reconstruction. This is the most common ligament reconstruction carried out by knee surgeons. This procedure is carried out approximately 1 million times per year globally.

Alternatively, a new technique has also been developed to allow reattachment of the ACL to its attachment point on the femur. This is called an ACL repair. Currently this operation has only been performed on a relatively small number of patients but is growing in terms of interest.

As shown in FIG. 2, the knee joint (9) is at the junction of the tibia (11) and the femur (10). The fibula (12) is not directly involved in the joint. Articular cartilage (13) covers the sections of the femur that come into contact with the tibia during movement of the joint. The ACL traverses across the knee joint in a diagonal trajectory from the central aspect of the knee or intercondylar notch. As shown in FIG. 3, the intercondylar notch (16) sits between the two large femoral condyles; the so-called medial femoral condyle (MFC) (14) on the inner aspect and the lateral femoral condyle (LFC) (15) on the outer aspect.

The ACL has a start point or origin on the femur and an insertion point on the tibia. As shown in FIG. 5, the ACL takes its origin at the very back of the intercondylar notch on the femur from two distinct points on the inner surface (17) of the lateral femoral condyle (15). The two main bundles are divided into the larger and to the right medial or AM bundle and the smaller and to the left posterolateral or PL bundle. (The origin of the AM bundle (19) and PM bundle (20) are shown in FIG. 5.) The ACL then traverses down onto the tibia to a comma-shaped insertion point which is placed at the front of the tibia just slightly on the inner or medial side.

For an ACL reconstruction the object of the exercise is to get good visualisation of the start point and finishing point of the ligament and to drill tunnels in the femur and the tibia at these two points to allow the passage of a new ACL graft that is then fixed into position using different types of fixation devices.

The failure rate of this procedure varies from 5% in those over 25 years old to 20% in those under 25 years old. The most common age group for injuring the ACL is the under 25s. With 1 million ACL procedures being carried out each year globally there are a significant number of failures numbering hundreds of thousands. The most common cause of failure of the ACL reconstruction surgery is mal-positioning of the tunnels, especially the tunnel in the femur (the femoral tunnel). Usually the mal-positioning places the tunnel high in the notch in a non-anatomic position. The main reason for this is relatively poor visualisation of this area due to the bony outline and constraints of the knee, which limit the ability to place the arthroscope in a position allowing good and effective visualisation. In particular, a surgeon using a front outer or anterolateral soft tissue portal (see FIGS. 1 and 3, in which the location of anterolateral portal is shown at (4)) views the position of the femoral tunnel by trying to look up the side wall of the intercondylar notch of the knee. The best way of describing this is to liken it to putting your face against a wall to see a picture which is hanging on the same wall. Attempts to improve this view have been made by looking from the front medial side or anteromedial portal (see FIGS. 1 and 3, in which the location of the anteromedial portal is shown at (3)), but this can be likened to taking your head slightly away from the wall. The view is improved but still restricted. The further problem then arises that the instruments also need to come in from the same area, making surgery difficult as the arthroscope and instruments are then very close to one another.

Another challenge posed by arthroscopy is associated with the extravasations or leakage of fluid into the soft tissues surrounding the joint. As mentioned above, arthroscopy or arthroscopy surgery often involves inflow and outflow of fluid into the joint to aid visibility and to clear any debris. Since there is no tight fit between the arthroscope and the soft tissue portal through which it is introduced into the joint, fluid inevitably leaks along the sides of the scope and into the surrounding soft tissues causing them to expand or swell, and making surgery progressively more difficult. This is especially relevant when a mechanical pump is used to drive the fluid in, which is common in knee, shoulder, and hip surgery. Where the swelling presses on the joint, this can interfere with the procedure and hinder visualisation. This is most commonly seen in the shoulder. Further, the swelling can also lead to complications such as oedema, compartment syndrome and respiratory compromise, which can sometimes be life-threatening.

### SUMMARY

The present disclosure accordingly provides in one aspect a method of obtaining an image of a target area in a joint of a subject with a device comprising an image capture component, said method comprising:

positioning at least a part of the device in an aperture in a bone involved in the joint such that the image capture component is in operable relation to the target area; and

obtaining an image of the target area with the image capture component.

In certain embodiments, the aperture in the bone may be a tunnel in the bone extending between a first point on an outer surface of the bone distal to the joint and a second point on a surface in the joint. In some embodiments, the method may further include forming the bone aperture. In certain embodiments, forming the bone aperture may include forming a tunnel in the bone involved in the joint. The bone tunnel may extend between a first point on an outer surface of the bone distal to the joint and a second point on a surface in the joint. The tunnel in the bone may be formed using intra-operative X-ray or CT imaging of the joint to aid positioning of the tunnel. In some embodiments, forming the tunnel in the bone is performed using an aiming device comprising a marking hook, wherein the method comprises placing a first end of the marking hook through a soft tissue portal onto the second point in the joint, and forming the bone tunnel from the first point to the second point using a guide wire or drill directed from the first point to the second point with the aiming device. The aperture in the bone may be formed using a guide wire followed by a reamer, or wherein the aperture in the bone is formed using a drill.

In certain embodiments, the image capture component may be positioned directly opposite the target area or is positioned at a point offset from a point directly opposite the target area. In certain embodiments, in the case that the image capture component is positioned at a point offset from a point directly opposite the target area, the image capture component has a line of sight to the target area that is at no more than 45° to a theoretical line between the target area and the point directly opposite the target area. The line of sight to the target area may be at no more than 20° to the theoretical line.

In some embodiments, positioning at least a part of the device in an aperture in a bone involved in the joint includes inserting the part of the device into the aperture in the bone and orientating the image capture component such that it has a line of sight to the target area. Certain examples may include, securing the part of the device positioned in an aperture in a bone involved in the joint such that the image capture component may be maintained in operable relation to the target area. The securing may include attaching the device to a third point on the bone adjacent to the first point.

In some embodiments, the image capture component may be orientated at between 0° to 90° to a longitudinal axis of the part of the device positioned in the aperture. The image capture component may include an aperture including a lens. The device may include an elongate portion having a first end and an opposing second end, wherein at least a part of the elongate portion is positioned in the aperture in the bone, and wherein the image capture component is at the first end. The device may include an illumination component at the first end which illuminates the target area and the image capture component comprises an aperture comprising a lens which captures light reflected from the target area. The device may include a camera at the first end positioned behind the lens. In some embodiments, the device may be an arthroscope.

In certain embodiments, the joint is a knee joint, a shoulder joint, a hip joint, or an elbow joint, or a joint of the wrist, spine, foot or ankle. The target area may be an area of soft tissue or an area of bone in the joint. The target area may be a ligament attachment site on a bone, joint surface cartilage, or a meniscus. In some embodiments, wherein the joint is a knee joint, the target area may be selected from an area within an intercondylar notch of a femur, an area at the top of a tibia, an area of meniscus, an area of a posterior meniscus, an area of an anterior meniscus, an ACL femoral footprint, an ACL tibial footprint, a PCL femoral footprint, a PCL tibial footprint, an area of a lateral surface of an intercondylar notch, and an area of joint surface cartilage. In some embodiments, wherein the joint is an ankle joint, and the target area may be an area of joint surface cartilage or an area of a talar dome. In particular embodiments, wherein the joint is a hip joint, the target area may be an area of a joint surface, a part of a ligamentum teres, or a part of an acetabular labrum. In certain embodiments, wherein the joint is a shoulder joint, the target area may be an area of a joint surface or a part of a glenoid labrum.

In particular embodiments, one or more of the steps may be performed robotically. The method of imaging may be performed as part of a method of surgery. The method of surgery may be a ligament repair or reconstruction, or a meniscal surgery. In some embodiments, the ligament may be an anterior cruciate ligament (ACL) or a posterior cruciate ligament (PCL).

A device comprising an image capture component is used to obtain an image of a target area of a joint in a subject. At least a part of the device is placed in a bone aperture such that the image capture component is in operable relation to the target area. In particular, an intraosseous aperture is utilised rather than a soft tissue portal. The advantage of the method is that the image capture component can be placed in novel positions in the joint in order to improve visualisation of target areas and obtain new views around the joint thus assisting the medical professional in their inspection of, and surgery on, the target area. Diagnosis and treatment can be improved as a result of a different or improved view of the target area being obtained. The intraosseous aperture can be achieved by drilling tunnels in the bone involved in the joint to accommodate an arthroscope. The bone aperture provides a rigid structure unlike a soft tissue portal and therefore helps to maintain the position of the image capture component once the device, or at least part thereof, has been inserted into the bone aperture. As such, the device is held more securely in place as compared to when the device is inserted into the joint through soft tissue portals, and the risk of soft tissue and joint damage is minimized Moreover, in some embodiments described below the device can additionally include a securing component to enable the device to be secured to the bone. As such, there is less (or no) movement of the device (the device may be effectively stationary or moving only very minimally) during the procedure, assisting the medical professional in maintaining the view of the target area, and reducing the risk of uncontrolled damage to soft tissue, joint structures or surfaces of the joint. Moreover, a close fit between the bone aperture and the device can be achieved. Thus, loss of fluid from the joint along the sides of the device and into the surrounding tissues can be minimized

These features are advantageous over the methods that utilise arthroscopes inserted into the joint via soft tissue portals to obtain visualization of the target area, the limitations of which are described above.

In another aspect, there is provided a method of minimally invasive surgery for ACL or PCL graft attachment to a target area on a femur or a tibia in a knee joint of a subject, wherein the method uses an arthroscope to obtain a view of the target area, the method comprising:

forming a bone tunnel in the femur or the tibia which extends between a first point on an outer surface of the femur or the tibia distal to the joint and a second point on a surface in the joint;

inserting a part of the arthroscope comprising an image capture component into the bone tunnel such that the image capture component is positioned in operable relation to the target area;

using the view of the target area obtained by the arthroscope to form a socket in the target area for the graft;

inserting an end of the graft into the socket; and

securing the graft in the socket.

In some embodiments for ACL graft attachment, the target area may be an ACL origin on a lateral surface of an intercondylar notch, and in forming a bone tunnel in the femur or the tibia which extends between a first point on an outer surface of the femur or the tibia distal to the joint and a second point on a surface in the joint, the bone tunnel is formed in the femur between the first point on an outer surface of the femur distal from the joint and the second point on a medial surface of the intercondylar notch. The target area may be a ligament origin on the tibia, and a bone tunnel may be formed in the femur between a first point on an outer surface of the femur distal from the joint and the second point in an upper part of an intercondylar notch.

In certain embodiments, forming a bone tunnel in the femur or the tibia which extends between a first point on an outer surface of the femur or the tibia distal to the joint and a second point on a surface in the joint is performed using an aiming device comprising a marking hook, wherein the method includes placing a first end of the marking hook through a soft tissue portal onto the second point in the joint, and forming the bone tunnel from the first point to the second point using a guide wire or drill directed from the first point to the second point with the aiming device. The drill may be configured as an outer sheath surrounding an obturator inserted into a lumen, wherein the method comprises withdrawing the obturator after the tunnel is drilled and inserting a part of the arthroscope comprising an image capture component into the bone tunnel such that the image capture component is positioned in operable relation to the target area, the part of the arthroscope comprising the image capture component may be inserted into the lumen of the outer sheath. In certain embodiments, the method may be a robotic method of surgery.

In a further aspect there is provided a device for use in a method of obtaining an image of a target area in a joint of a subject, the device comprising:
an elongate portion having a first end and an opposing second end;
a bone cutting element at the first end for creating a bone tunnel;
a lumen surrounded by the bone cutting element at the first end, the lumen extending from the first end to the opposing second end; and
a scope positioned in the lumen, wherein the scope include an image capture component at the first end.

In some embodiments, the scope is rotatably disposed in the lumen and includes a mechanism for reversibly securing the scope within the lumen. The scope may be removable from the lumen and can be replaced by an obturator. In particular embodiments, the device can further include a connector portion positioned at the second end for connecting the device to one or more of a power source, a light source, a computer, a drill guide, a camera, a rotation mechanism, and a screen, or wherein the device comprises one or more of a power source, a computer, a drill guide, a camera, a rotation mechanism, or a screen. The second end may be joined to a handle. In certain embodiments, the device may include a securing part for reversibly securing the position of the device in the bone tunnel. The scope may include a first optical channel for transmitting light from a light source at the second end to an illumination component at the first end and a second optical channel for transmitting the light captured by the image capture component to the second end. The scope may include a first optical channel for transmitting light from a light source at the second end to an illumination component at the first end, wherein the image capture component at the first end comprises a camera positioned behind a lens, and wherein the scope comprising an electrical channel connecting the camera to the second end. In particular embodiments, the image capture component comprises a lens or a lens system. The bone cutting element may be selected from a reamer or a drilling element. The drilling element may form an outer sheath to the elongate portion. The elongate portion may have a width or diameter of less than 10 mm The first end may include a removable or retractable cover for the image capture component. The device may include a fixation component for securing the device in position in the bone tunnel with the image capture component in operable relation to the target area. The elongate portion or the lumen may include an inflow channel and an outflow channel to allow fluid to be introduced into and removed from the joint.

In a still further aspect there is provided a method of inserting a scope into a joint of a subject in order to obtain an image of a target area in the joint, the method comprising forming a bone tunnel using the bone cutting element of the device, and inserting the scope into the lumen of the device. The method may include securing the scope within the device.

Embodiments of apparatuses, methods, and systems for visualizing a target area of a joint during arthroscopic joint inspection and joint surgery such as those disclosed herein may also be used in combination or in addition with those described in U.S. patent application Ser. No. 15/234,999, filed Aug. 11, 2016, published as U.S. 2017/0042408 A1 on February 16, 2017, issued as U.S. Pat. No. 10,405,886 on Sep. 10, 2019, titled “FULLY INTEGRATED, DISPOSABLE TISSUE VISUALIZATION DEVICE,” the disclosure of which is hereby incorporated by reference herein in its entirety. Including further details relating to embodiments of devices for visualizing tissue.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

### DESCRIPTION OF THE FIGURES

Embodiments of the present disclosure will now be described by way of example only with reference to the accompanying drawings, in which:

FIG. 1 shows a schematic of flexed knee (1) of a patient viewed from the front, i.e. looking straight at the knee, showing the locations of the arthroscopic soft tissue portals already established for the knee. The dashed line markings on the knee represent: the location of the knee cap (2); the location of the patella tendon (7); the joint line (8); the location of the (central) anterolateral soft tissue portal (4); the location of the high anterolateral soft tissue portal (6); the location of the anteromedial soft tissue portal (3); and the location of the high anteromedial soft tissue portal (5).

FIG. 2 is a drawing of a knee joint (9) showing the arrangements of the bones in the knee with the knee fully extended. (No ligaments are shown.) The arrows show: the femur (10); the tibia (11); the fibula (12); and the articular cartilage (13).

FIG. 3 is a drawing of a knee joint (9) showing the arrangements of the bones in the knee with the knee flexed. (No ligaments are shown.) The arrows show: the femur (10); the tibia (11); the medial femoral condyle (14); the lateral femoral condyle (15); and the intercondylar notch (16). The circles marked with dashed lines show the approximate locations of the entry of the anterolateral soft tissue portal (4) and the anteromedial soft tissue portal (3) into the joint.

FIG. 4 is a drawing of a knee joint with the knee flexed to illustrate how the lateral femoral condyle (15) limits the view of the inner surface of the lateral femoral condyle when the arthroscope is inserted through the anterolateral portal. Also marked are the medial femoral condyle (14), the articular cartilage (13) and the intercondylar notch (16).

FIG. 5 is a drawing showing a section (18) through the femur at the lateral femoral condyle (15). The location of the origins of the anterior cruciate ligament (ACL) on the inner surface (17) of the lateral femoral condyle are marked with dashed circles, in which the dashed circle (19) represents the origin of the anteromedial (AM) bundle of the ACL and the dashed circle (20) represents the origin of the posterolateral (PL) bundle of the ACL.

FIG. 6 is a drawing showing the position of a bone tunnel (21) formed between a first point (22) on the outer surface of the bone and a second point (23) on a surface in the knee joint (9) in an embodiment. Also marked on the drawing are: the tibia (11); the medial femoral condyle (14); the medial outer surface of the femur (30); the medial surface (31) of the intercondylar notch; the intercondylar notch (16); and the lateral femoral condyle (15).

FIG. 7 is a drawing showing how the bone tunnel may be formed between a first point (22) on the outer surface of the bone and a second point (23) on a surface in the knee joint (9) using an aiming device/jig (24). Also marked on the drawing are: the tibia (11); the marking hook (25) of the aiming device/jig (24); the guide part (26) of the aiming device/jig (24); and the drill (27). The large arrow marks the direction in which the drill (27) is guided into the bone by the guide part (26).

FIG. 8 is a drawing showing a step in the method using the device according to an embodiment in which the drill head (28) has been used to form the bone tunnel and the elongate portion (32) is positioned in the bone tunnel in the medial femoral condyle, extending from the medial outer surface (30) of the femur to the medial surface (31) of the intercondylar notch. Also shown is the connector portion (33) of the device, the groove (34) in the connector portion; and the lumen (29) where the image capture component may be positioned.

FIG. 9A and 9B are drawings showing parts of an arthroscope. FIG. 9A shows an arthroscope sheath comprising an elongate part (36) that is suitable for insertion into a soft tissue portal, and parts arranged for fluid inflow (37) and fluid outflow (38). FIG. 9B shows an obturator (39) comprising a blunt end (40) and a handle (41). The obturator can be inserted into the arthroscope sheath such that the sheath can be used to establish the soft tissue portal. Thereafter the obturator can be removed and replaced by the part carrying a light and a camera, which can be used to capture an image of the joint.

FIG. 10 is a drawing showing parts of the device in an embodiment. The device may include an elongate portion (32) with a first end (48) and a second end (49). The elongate portion is a drill sleeve with a drill head (28) at the first end. The drill sleeve is cannulated and has a lumen (29). At the second end the device may include a connector portion (33). The connector portion includes a groove (34) to assist in connection to a drill connector (42) or to an equipment connector (43).

FIG. 11A is a drawing showing the arrangement of one end of the connector portion (33) showing the lumen (29) the groove (34) and the sloping sections (47).

FIG. 11B is a drawing showing the arrangement of one end of the drill connector (42) which is to engage with the surface of the connector portion (33) shown in FIG. 11A. FIG. 11B shows the central cavity (46), ridge sections (44), the lumen (29) and the raised lip (45) of each ridge section.

### DETAILED DESCRIPTION

Various embodiments are described in detail and may be further illustrated by the provided Figures. As used in the description herein and throughout the claims that follow, the meaning of “a,” “an,” and “the” includes plural reference unless the context clearly dictates otherwise.

The terms used in this specification generally have their ordinary meanings in the art, within the context of the present disclosure, and in the specific context where each term is used. Certain terms that are used are discussed below, or elsewhere in the specification, to provide additional guidance to the practitioner in describing the present methods and devices.

As described above, the present disclosure provides a method of obtaining an image of a target area of a joint in a subject with a device comprising an image capture component, said method comprising: positioning at least a part of the device in an aperture in a bone involved in the joint such that the image capture component is in operable relation to the target area; and obtaining an image of the target area with the image capture component.

The method is a method for visualising a joint which is suitable for use in medical procedures in which it is not possible or desirable to open up the joint in order to obtain a direct view. In particular, the method is suitable for use in keyhole or minimally invasive surgeries, where arthroscopes are normally used. The joint may be a knee, shoulder, hip, or elbow joint, or a joint of the wrist, spine, foot or ankle. In particular, the joint may be a joint in which it is already known to utilise an arthroscope. Optionally, the joint may be selected from a knee, shoulder or hip joint. Further optionally, the joint may be a knee joint.

The target area of the joint is the specific area that the medical professional wishes to obtain a view of in order to inspect the joint or perform a surgical procedure on the joint. The target area can be interosseous, i.e. located in an area between the two or more bones that meet at the joint, and/or may include an area of soft tissue or a bony area of the joint. In particular, the target area may include a joint surface or structure, such as cartilage, bone, ligament, meniscus, or synovial capsule.

The target area may include a ligament attachment site or origin, joint surface cartilage or a meniscus. In particular, where the joint is a knee joint the target area may be selected from the posterior cruciate ligament (PCL) origin or footprint on the femur, the PCL origin or footprint on the tibia, the ACL origin or footprint on the femur, the ACL origin or footprint on the tibia, the intercondylar notch, the lateral surface of the intercondylar notch, and the medial surface of the intercondylar notch. The target area may also be along the length of the medial or lateral menisci, especially the root of the medial or lateral menisci at the back of the joint which is currently difficult to visualise with soft tissue portals, or the front attachments which again can be difficult to see with traditional arthroscopic viewing via soft tissue portals. In particularly tight knee joints (i.e. where the space between the femur and tibia is tight) it is difficult to see the meniscus through its entire length with soft portal viewing.

Further, where the joint is the ankle joint, the target area can be an area of joint surface cartilage or an area of the talar dome. Where the joint is the hip joint, the target area can be an area of the joint surface, a part of the ligamentum teres, or a part of the acetabular labrum. Where the joint is the shoulder joint the target area can be an area of the joint surface or a part of the glenoid labrum.

A device comprising an image capture component is to be used in the method and can be classed as an endoscope or more specifically an arthroscope. In particular, such devices are those which are designed to be inserted or placed directly into a part of the body (with the minimum amount of damage to body tissues and structures) in order to obtain images without the need for cutting the body part open. The image may be a single image or a plurality of images. In particular examples, the image is a moving image, such that a medical professional can “see” inside the joint.

The device utilises an imaging modality, such as light or ultrasound, in order to obtain an image of the target area. Therefore, the device may include an image capture component to capture the image of the target area by capturing signals reflected back from the target area. The device may also include a signal emitter which emits a signal into the joint.

In one embodiment the signal emitter emits light which illuminates the target area and light reflected back from the target area is captured via an image capture component that includes an aperture and a lens or lens system.

In one embodiment the device may be an arthroscope comprising a rigid or flexible tube, a light source to illuminate the target area, an optical fibre system to deliver light to illuminate the target area and to transmit captured light reflected back from the target area outside the body. (Where the scope includes a rigid tube a lens system may be used to transmit captured light back.) The image from the reflected light may be viewed with an eyepiece or a camera. Alternatively the image capture component may include a camera adjacent to the lens, such as a CMOS or CCD camera, which capture the image and transmit an electrical signal. The device may be connected to a screen or computer to display the image or transmit the image to a medical professional at a different location.

In an alternative embodiment the device may be a capsule which is inserted or placed in the bone aperture and which may include a light source, such as one or more light emitting diodes, which emit light, an image capture component comprising a lens to guide the reflected light and a component, such as for example a photodiode, to convert the captured light into an electrical signal. The device also may include an antenna and a transmitter system to transmit signals based on the captured image. The signals can be picked up by external sensors connected to a screen or computer for display of the captured images.

Some embodiments may include one or more of the devices for visualizing tissue as described in U.S. patent application Ser. No. 15/234,999, filed Aug. 11, 2016, published as U.S. 2017/0042408 A1 on Feb. 16, 2017, issued as U.S. Pat. No. 10,405,886 on Sep. 10, 2019, titled “FULLY INTEGRATED, DISPOSABLE TISSUE VISUALIZATION DEVICE.” One of skill in the art will understand that the devices described in U.S. Pat. No. 10,405,886 are merely examples of possible visualization devices and tools that may be used with the embodiments described herein.

In the method the device or at least a part of the device comprising the image capture component is positioned so that the image capture component is in operable relation to the target area. This means that the image capture component is able to capture an image of the target area, and in particular to capture signals reflected back from the target area. Where the target area is illuminated and the signals are light reflected back from the target area, the image capture component must have a line of sight to the target area, or be able to achieve a line of sight to the target area when the joint is manipulated during the inspection and/or surgery.

The image capture component can be positioned directly opposite the centre of the target area, such that it provides a view straight onto the target area, or at a point offset from a point directly opposite the target area, such that the view provided is more oblique. This provides a new way of looking at target areas or areas of interest within joint cavities and provides new views not previously achievable with soft tissue portals. When the image capture component is positioned at a point offset from the position opposite the target area the image capture component has a line of sight to the target area that is at no more than 45°, or optionally no more than 20°, to a theoretical line between the target area and the point directly opposite the target area.

Moreover, as described further below, different views can be obtained with the method using image capture components that are positioned within the device at an angle. As described above, it is known in the art to provide arthroscopes with angled tips, e.g. at 30°, and a device with a similarly angled image capture component can be utilised in the method. In particular, the image capture component can be angled at from 0° to 90° to the longitudinal axis of the part of the device positioned in the aperture. The image capture component may also be moveable either with the device or within the device in order for the view to be changed once the device is in position. This may be achieved with a device that utilise flexible fibre optic scopes.

According to the method, the device or at least a part of the device comprising the image capture component is positioned in an aperture in a bone involved in the joint. For example, where the joint is the knee the device or at least part of the device can be positioned in a bone aperture in the tibia or femur, or where the joint is the shoulder the device or at least part of the device can be positioned in a bone aperture in the humerus or the glenoid shoulder blade.

The bone aperture may be a socket sized to fit the device, for example a hole or blind tunnel that has one closed end and one end that opens into the joint.

Alternatively, as shown in FIG. 6, the bone aperture may be a tunnel with two open ends, extending from a first point (22) on the outer surface of the bone distal to the joint to a second point (23) on a surface in the joint. For example, where the bone aperture is in the medial surface (31) of the intercondylar notch (16) on the femur the tunnel may extend to a medial outer surface (30) of the femur.

The aperture in the bone may have an approximately circular cross section. The diameter of the aperture depends to a certain extent on the size and nature of the bone. The diameter of the aperture may be as small as practicable in order to minimize unnecessary damage to the bone and surfaces of joint. In some embodiments the diameter is less than 10 mm, or less than 5 mm Alternatively, the aperture may be 1.0 to 10 mm, 1.5 to 10 mm, 1.8 to 5 mm, 3.5 to 4.5 mm or 5 to 10 mm in diameter.

The method may include forming the bone aperture. In particular, this may include forming a bone tunnel in the bone involved in the joint. This may be performed using the device described herein, which is discussed further below, or may be performed using a device already known in the art, such as those currently utilised to form bone tunnels for ligament attachment.

The medical professional may utilise arthroscopic imaging via one or more soft tissue portals in order to determine a suitable location for the bone aperture. They may also utilise one or more soft tissue portals in the formation of the bone aperture. (The formation of soft tissue portals is discussed further below.)

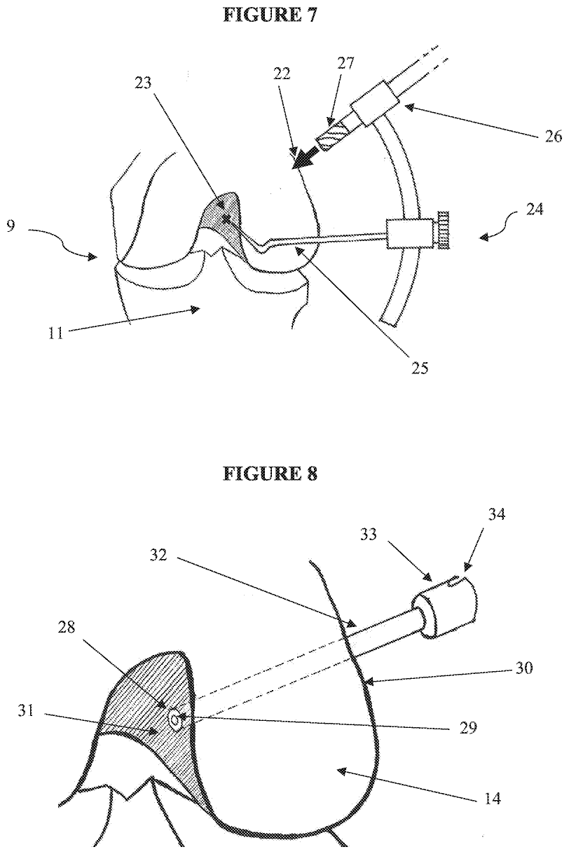

In one embodiment, as shown in FIG. 7, where the bone aperture is a tunnel with two open ends that extends between a first point (22) on the outer surface of the bone distal to the joint and a second point (23) on a surface in the joint, the bone tunnel may be formed using an aiming device/jig (24) comprising a marking hook (25), which is inserted into the joint via a soft tissue portal. The marking hook (25) is a rigid surgical instrument comprising a curved distal tip at a first end and an opposing second end. The tip at the first end can be placed into the joint, through a soft tissue portal, onto the second point (23) on the surface in the joint in order to mark the location where the bone tunnel is to exit the bone within the joint. The second end of the marking hook (25), which extends outside the body of the subject, is attached to the rest of the aiming device/jig. The aiming device/jig may include a guide part (26), such as a guide sleeve, into which a drill or guide wire can be inserted. The configuration of the aiming device/jig allows a drill (27) or guide wire inserted into the bone via the guide part (26) to be directed to the second point (23) on the surface in the joint, which is marked by the first end of the marking hook (25).

In another embodiment the bone tunnel is formed using computer-generated 3D images of the joint in order to ensure that the tunnel is placed in the correct position.

The bone aperture, and in particular the bone tunnel, may be formed using a drill with or without the use of a guide wire. Alternatively, the tunnel may be formed using a guide wire and a reamer.

The methods described above may have one or more steps performed robotically and/or using computer generated 3D images of the joint, in order to guide the positioning of the image capture component and/or the forming of the bone aperture. In particular, the bone aperture can be formed using intra-operative X-ray MRI or CT imaging of the joint to assist in aperture placement. Information regarding the arrangement of a particular joint based on these imaging methods can be provided to a suitably programmed computer. Based on this information an ideal tunnel position can be selected and drilling performed robotically.

As highlighted above, the method of imaging can be performed during surgery, and in particular to provide an image of the area on which the surgery is to be performed. The surgery may be anything that can be done to the soft tissues inside a joint, including ligament repair or reconstruction, or meniscal surgery. In the knee for example the surgery may be ligament repair or reconstructions. Examples of these ligaments are the anterior cruciate ligament (ACL) or a posterior cruciate ligament (PCL) of the knee. Other surgeries are those that address talar dome injuries in the ankle, which is not infrequently damaged and is difficult for the surgeon to see using soft tissue portals, triangular fibrocartilage complex (TFCC) surgeries in the wrist, and meniscal surgery, involving either suturing the body of the meniscus or its attachment points.

As indicated above, the method of imaging is advantageous in methods of surgery as currently joint visualisation with soft tissue portals is often restricted. Moreover, there is only a limited space between the ends of the bone and visualisation during surgery can be compromised as a result.

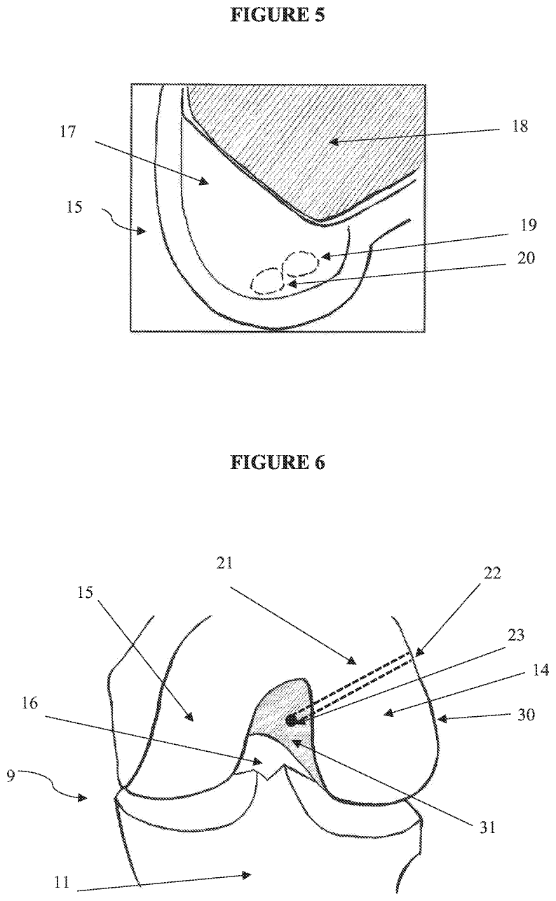

According to the present disclosure, where, for example, surgery is reconstruction or repair of the ACL or the PCL the target area may be selected from the following: the origin of the ACL on the femur, the origin of the ACL on the tibia, the origin of the PCL on the femur, or the origin of the PCL on the tibia. For example, where the target area is the origin of the ACL on the inner surface (17) of the lateral femoral condyle (15), which is shown in FIG. 5 (as the marked areas (19) and (20)), the second point (23) on the surface in the joint can be on the medial surface (31) of the intercondylar notch (16) as shown in FIG. 6.

In an embodiment, the method of imaging can be used in a method of minimally invasive ACL or PCL graft attachment to a target area on a femur or a tibia in a knee joint of a subject.

In particular, such a method can use an arthroscope to obtain a view of the target area, the method comprising:

forming a bone tunnel in the femur or the tibia which extends between a first point on an outer surface of the femur or the tibia distal to the joint and a second point on a surface in the joint;

inserting a part of the arthroscope comprising an image capture component into the bone tunnel such that the image capture component is positioned in operable relation to the target area;

using the view of the target area obtained by the arthroscope to form a socket in the target area for the graft;

inserting an end of the graft into the socket; and

securing the graft in the socket.

When inserting a part of the arthroscope comprising an image capture component into the bone tunnel such that the image capture component is positioned in operable relation to the target area, the image capture component may be positioned at the second point on the surface of the joint or may be positioned beyond the surface of the joint and in the joint space.

In one embodiment, where the target area is an ACL origin on a lateral surface of an intercondylar notch, the bone tunnel can be formed in the femur between the first point (22) on an outer surface of the femur distal from the joint and the second point (23) on a medial surface (31) of the intercondylar notch (see FIG. 6).

In a further embodiment, where the target area is a ligament origin on the tibia, the bone tunnel in can be formed in the femur between a first point on an outer surface of the femur distal from the joint and the second point in an upper part of an intercondylar notch.

The steps of forming a bone tunnel and inserting the part of the arthroscope comprising an image capture aperture is as defined above for the method of imaging.

As described above, soft tissue portals can be used to determine the best location for the bone tunnel and/or be utilised to assist during forming the bone tunnel.

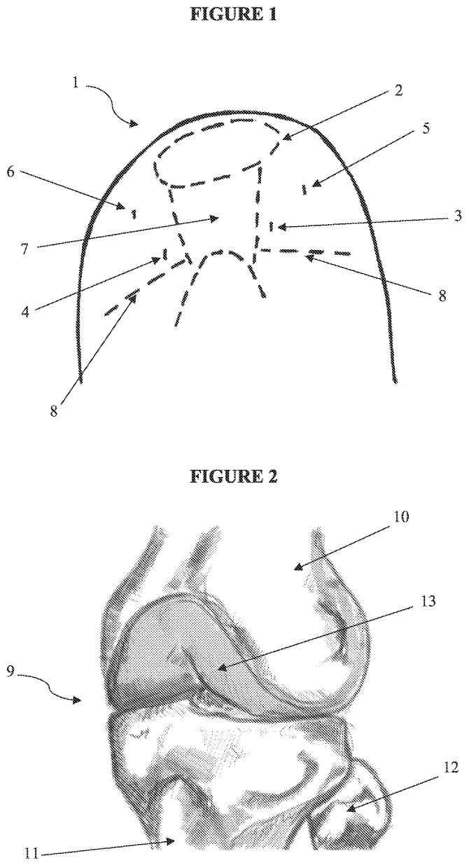

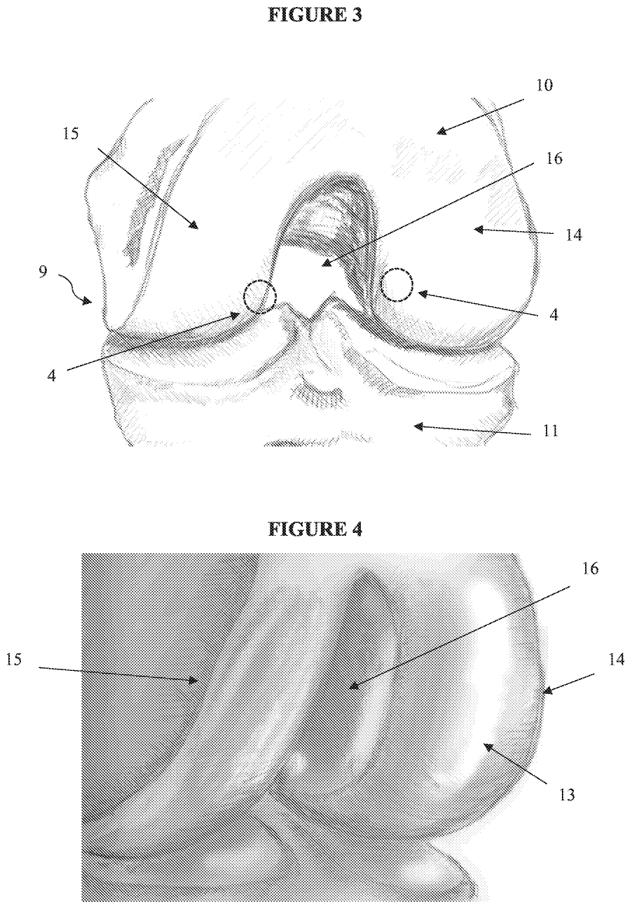

Suitable soft tissue portals that can be utilised in the formation of a bone tunnel for forming a tunnel on the medial side of the intercondylar notch (such that the image capture component would be facing the ACL femoral footprint) are the anterolateral portal, which can be used for viewing, and the anteromedial portal, which can be used to provide access for the marking hook of the aiming device/jig. The locations of the anterolateral portal (4) and the anteromedial portal (3) are shown in FIG. 1. Specifically, for the anterolateral portal the surgeon uses external palpation to define a safe triangle to allow the first arthroscopic portal to be made. This triangle is made up of the bony outline of the lateral femoral condyle on one side of the triangle, and then more medially (or towards the inner aspect) the patella tendon with the edge of this structure being palpated and noted. The bottom of the triangle is made up by the edge of the tibia or shinbone. Centrally within this triangle is a window that allows the surgeon to make the anterolateral portal. The skin is incised with a blade in a safe direction towards the notch. This skin incision may be approximately a centimetre in length. This is then widened slightly and the telescope is introduced. A telescope 4.5 mm in diameter including the outer sheath and with a tip that has a 30° angulation can be used.

Once the telescope is advanced beyond the soft tissues and the fat pad the anteromedial portal can then be created. Typically this can be done using the telescope inserted into the anterolateral portal by first palpating externally and then introducing a needle. Once the direction of the needle and position is considered adequate the knife can be introduced to create a second stab and allow introduction of the marking hook of the jig/aiming device.

After the marking hook has been inserted it can be used to mark the desired location on the medial side of the intercondylar notch. Externally, the marking hook is attached to the rest of the aiming device/jig, which, together with a drill or guide wire, can be used to form the tunnel as described above.

In particular, as described above the tunnel can be formed using the drills/guide wires/reamers as described above. Alternatively, the tunnel can be formed using the device of the present disclosure described below.

In particular in a further aspect, there is provided a device for use in a method of obtaining an image of a target area in a joint of a subject. The device may include:
an elongate portion having a first end and an opposing second end;
a bone cutting element at the first end for creating a bone tunnel;
a lumen surrounded by the bone cutting element at the first end, the lumen extending from the first end to the opposing second end; and
a scope positioned in the lumen comprising an image capture component at the first end.

The device is intended for use in the method of imaging described herein and is designed both to form the bone tunnel using the bone cutting element and be capable of capturing images using the scope. In particular the bone tunnel can be formed with the scope positioned within the lumen. Alternatively, the bone tunnel can be formed with an obturator inserted within the lumen. Once the bone tunnel is formed the obturator can be removed and the scope inserted. Once the image capture component of the scope is positioned in operable relation to the target area, images of the target area can be captured.

The elongate portion is intended to be inserted into the bone tunnel as this is being formed by the bone cutting element at the first end, and is sized accordingly. Once the tunnel is formed the elongate portion is sized to extend from the bone aperture at the joint surface through the bone tunnel and out of the open second end of the tunnel, such that the second end projects externally from the bone as shown in FIG. 8. In this way images captured by the image capture component can be relayed along the elongate portion outside the body and displayed on a screen or computer.

The elongate portion may be circular or substantially circular in cross-section. The maximum diameter of the elongate portion depends on the bone and the subject in which it is to be used. A smaller diameter may be chosen to limit damage to the bone. The diameter could be up to 20 mm but can be 10 mm or less, such as between 1.5 to 10 mm, or between 1.8 and 5 mm.

The elongate portion may include a bone cutting element at the first end. The bone cutting element is configured to form the bone tunnel. The bone cutting element may be a rotatable bone cutting element and may be selected from a reamer or a drilling element. Where the cutting element is a reamer the elongate portion may include a channel for accommodating a guide wire such that the tunnel can be formed along a line marked by the guide wire. The bone cutting element may be a drill.

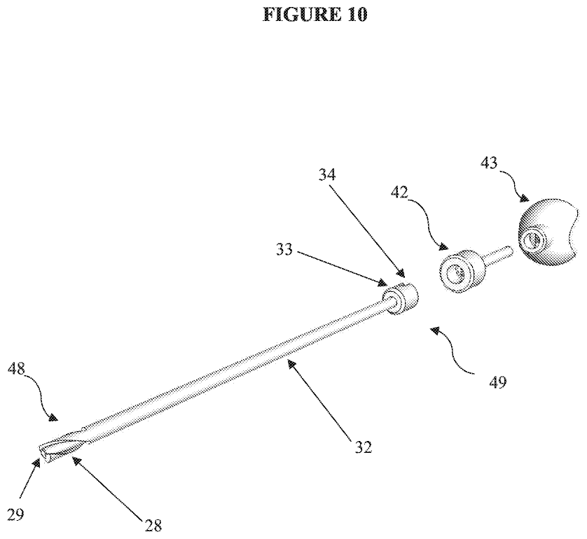

In an embodiment shown in FIG. 10 the elongate portion (32) is a drill sheath comprising a drill head (28) at the first end, the sheath comprising a lumen (29) that extends from the first end (48) to the second end (49).

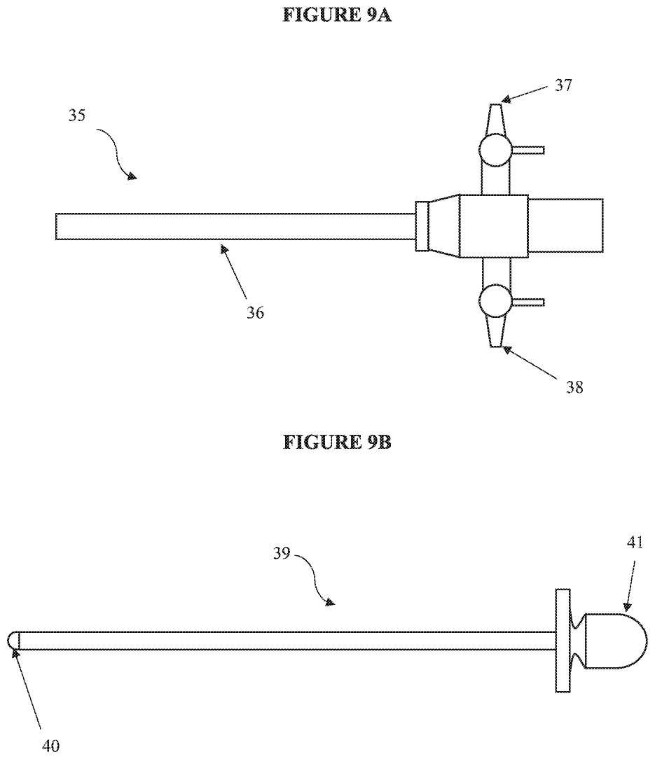

The elongate portion may include a lumen, optionally a central lumen, that extends from the first end to the second end. The lumen can be circular or substantially circular in cross-section. The lumen is intended to provide the “portal” into the joint and is sized to accommodate at least the scope (the sizes of which are described herein in relation to the method of imaging). As described above, while the bone tunnel is being formed the lumen may be blocked by an obturator (such as that shown as part (39) in FIG. 9B). Once the bone tunnel is formed the obturator can be removed from the lumen and the lumen used to provide access for the scope once the entry point into the joint space has been established.

Alternatively, the scope may be retained in the lumen. In this embodiment the first end of the elongate portion may include a removeable or retractable cover for the image capture component, such that this is protected while the tunnel is being formed but can be revealed once the first end is in position in the joint.

The lumen or the elongate portion may include an inflow channel and an outflow channel to allow fluid to be introduced into and removed from the joint. Alternatively, or in addition, the lumen or elongate portion may include one or more working channels through which surgical instruments may be inserted.

The scope part of the device may include an image capture component which is intended to be placed in operable relation to the target area once the bone tunnel has been formed, so as to be able to capture an image of the target area. In particular, the image capture component may include an aperture and a lens or lens system or a camera positioned behind a lens. This camera may be a CMOS or CCD camera. The image capture component may be orientated within the scope such that it is angled with respect to the longitudinal axis of the elongate portion, as described elsewhere herein. The angle can be from more than 0° to 90°, and may also be 30°.

The scope may include a first optical channel for transmitting the light captured by the image capture component to the second end. Alternatively, where the image capture component may include a camera positioned behind a lens, the scope may include a connection which connects the output from the camera with the second end.

The elongate portion or the scope may further include a second optical channel for transmitting light from a light source at the second end to an illumination component at the first end in order to illuminate the target area. Alternatively, the elongate portion or scope may include a light source at the first end.

The optical channels may include optical fibres or a lens system.

The device used in the methods described herein may be one or more of the devices for visualizing tissue as described in U.S. patent application Ser. No. 15/234,999, filed Aug. 11, 2016, published as U.S. 2017/0042408 A1 on Feb. 16, 2017, issued as U.S. Pat. No. 10,405,886 on Sep. 10, 2019, titled “FULLY INTEGRATED, DISPOSABLE TISSUE VISUALIZATION DEVICE,” the devices of which may be used advantageously be used in both arthroscopic portals, hard tissue, and/or soft tissue portals.

The scope part of the device may include arthroscopes.

The scope may be rotatable disposed in the lumen such that the position of the image capture component can be adjusted. This is particularly advantageous where the image capture component is angled with respect to the first end. In one example, a medical professional looking at the target area may wish to move the joint during inspection and surgery. By rotating the scope within the lumen the image capture component can be repositioned to the same degree, thus maintaining the view of the target area. The device may include a ratchet mechanism in order to allow the rotation. The device may further include a locking mechanism to secure the scope within the lumen once the desired view of the target area is achieved.

The scope may be inserted within the elongate portion such that the image capture component is at the first end of the elongate portion. However, in one embodiment the scope may extend beyond the first end of the elongate portion, such that during operation of the device the image capture component can be moved further into the joint, beyond the first end, to achieve further views of the joint, or retracted back into the lumen, during the course of the joint inspection or surgical procedure. This movement may also be controlled by a ratcheting mechanism and/or a locking mechanism in the device which controls movement of the scope.

The positioning of the device within the bone tunnel restricts the movement of the device such that movement of the device and scope is restricted and much more easily controlled by the mechanisms described above. This opens up the option for robotic assistants to operate the device and reduces the need for skilled human operators.

Moreover, the arrangement of the device, with the cutting element retained in place during imaging, ensures a tight fit with the bone tunnel and prevents leakage of fluid out of the joint along the sides of the device.

The second end of the elongate portion may optionally be joined to a handle.

The device may further include a connector portion which is shown as part (33) in FIG. 10. The connector portion is to be used to join the second end (49) of the elongate portion to a variety of working elements that will enable full operation of the device. The connector portion may also engage with the scope. The connector portion may allow the elongate portion/scope to be connected to one or more additional pieces of equipment, such as a power source, a light source, a computer, a screen, a rotation mechanism to drive rotation of the drill, and a drill guide. Alternatively, the device may include one or more of a power source, a light source, a computer, a screen, a rotation mechanism to drive rotation of the drill, or a drill guide.

In an embodiment shown in FIG. 10 the connector portion (33) may include a groove (34) for connecting with a drill connector (42) in order to drive rotation of the elongate portion (32) and drill head (28). The same connector portion (33) can also engage with an equipment connector (43).

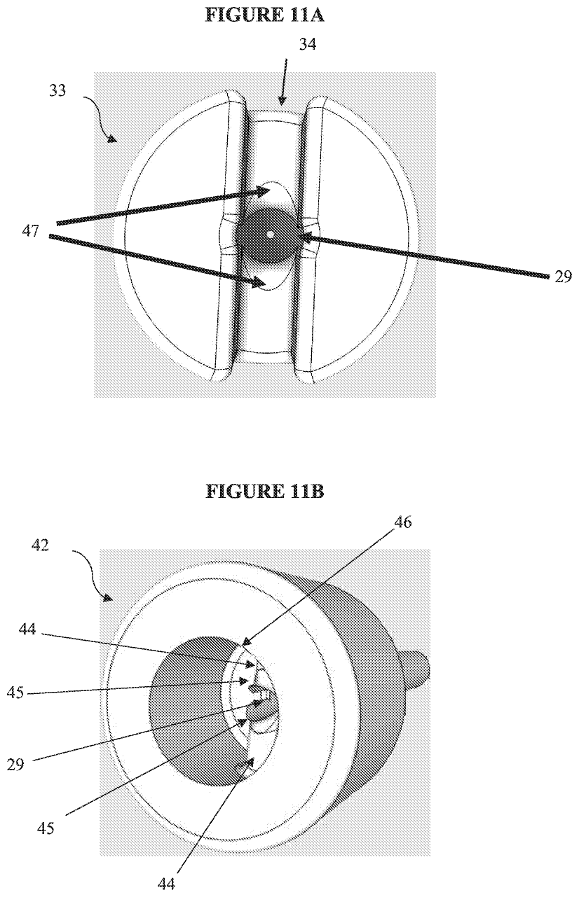

An example of a suitable connection mechanism in an embodiment is shown in FIGS. 11A and 11B. In particular, an end surface of the connector portion (33) equipped with a groove is shown in FIG. 11A. The connector portion also has a lumen (29) to connect to the lumen of the elongate portion. The groove (34) is straight and extends from one side of the end surface to the other. Where the groove connects with the lumen sloping sections (47) are provided to assist engagement. An end surface of a drill connector (42) is shown in FIG. 11B which is configured to engage with the end surface of the connector portion shown in FIG. 11A. The end surface of the drill connector may have a central cavity (46) to accommodate the end surface of the connector portion. Two ridge sections (44) are positioned in the central cavity (43) either side of the lumen (29) and are arranged to engage with the groove and lumen of the end surface of the connector portion. Where each ridge meets the lumen is a raised lip (45) which engages with the sloping section (47) of the end surface of the connector portion.

The device may further include a fixation component for securing the device in position in the bone tunnel with the image capture component in operable relation to the target area.

In a further aspect there is provided a method of inserting a scope into a joint using the device described above in which an obturator is positioned in the lumen. In particular, the method includes forming a bone tunnel using the bone cutting element of the device, removing the obturator and inserting the scope. The method may further include securing the scope, once it is positioned within the elongate portion, with the image capture component in operable relation to the target area. The method may further include rotating the scope within the elongate portion and/or inserting the scope through the elongate portion such that the image capture component is positioned beyond the first end of the elongate portion.

Also envisaged as part of the present disclosure are kits comprising: (i) an elongate portion equipped with a bone cutting element and a lumen as described above; (ii) a scope comprising an image capture component as described above; and (iii) an obturator suitable for insertion into the lumen.

Other variants or use cases of the disclosed techniques may become apparent to the person skilled in the art once given the disclosure herein. The disclosure is not limited by the described embodiments but only by the accompanying claims.

1.-31. (canceled)

32. A method of minimally invasive surgery for ACL or PCL graft attachment to a target area on a femur or a tibia in a knee joint of a subject, wherein the method uses an arthroscope to obtain a view of the target area, the method comprising:
forming a bone tunnel in the femur or the tibia which extends between a first point on an outer surface of the femur or the tibia distal to the knee joint and a second point on a surface in the knee joint;
inserting a part of the arthroscope comprising an image capture component into the bone tunnel such that the image capture component is positioned in operable relation to the target area;
using the view of the target area obtained by the arthroscope to form a socket in the target area for a graft;
inserting an end of the graft into the socket; and
securing the graft in the socket.

33. The method according to claim 32 for ACL graft attachment, wherein the target area is an ACL origin on a lateral surface of an intercondylar notch, and forming the bone tunnel comprises forming the bone tunnel in the femur between the first point on an outer surface of the femur distal from the knee joint and the second point on a medial surface of the intercondylar notch.

34. The method according to claim 32, wherein the target area is a ligament origin on the tibia, and forming the bone tunnel is formed in the femur between a first point on an outer surface of the femur distal from the knee joint and the second point in an upper part of an intercondylar notch.

35. The method according to claim 32, wherein forming a bone tunnel comprises using an aiming device comprising a marking hook, wherein the method comprises placing a first end of the marking hook through a soft tissue portal onto the second point in the knee joint, and forming the bone tunnel from the first point to the second point using a guide wire or drill directed from the first point to the second point with the aiming device.

36. The method according to claim 35, wherein the drill is configured as an outer sheath surrounding an obturator inserted into a lumen, wherein the method comprises withdrawing the obturator after the bone tunnel is drilled and inserting the image capture component is inserted into the lumen of the outer sheath.

37. The method according to claim 32, the method being a robotic method of surgery.

38. A device for use in a method of obtaining an image of a target area in a joint of a subject, the device comprising:
an elongate portion having a first end and an opposing second end;
a bone cutting element at the first end for creating a bone tunnel;
a lumen surrounded by the bone cutting element at the first end, the lumen extending from the first end to the opposing second end;
a scope positioned in the lumen, wherein the scope comprises an image capture component at the first end; and
wherein the elongate portion or the lumen comprises an inflow channel and an outflow channel to allow fluid to be introduced into and removed from the joint.

39. The device according to claim 38, wherein the scope is rotatably disposed in the lumen and comprises a mechanism for reversibly securing the scope within the lumen.

40. The device according to claim 38, wherein the scope is removable from the lumen and can be replaced by an obturator.

41. The device according to claim 38, comprising a connector portion positioned at the opposing second end for connecting the device to one or more of a power source, a light source, a computer, a drill guide, a camera, a rotation mechanism, and a screen, or wherein the device comprises one or more of a power source, a computer, a drill guide, a camera, a rotation mechanism, or a screen.

42. The device according to claim 38, wherein the opposing second end is joined to a handle.

43. The device according to claim 38, wherein the device comprises a securing part for reversibly securing the a position of the device in the bone tunnel.

44. The device according to claim 38, wherein the scope comprises a first optical channel for transmitting light from a light source at the opposing second end to an illumination component at the first end and a second optical channel for transmitting the light captured by the image capture component to the opposing second end.

45. The device according to claim 38, wherein the scope comprises a first optical channel for transmitting light from a light source at the opposing second end to an illumination component at the first end, wherein the image capture component at the first end comprises a camera positioned behind a lens, and wherein the scope comprising an electrical channel connecting the camera to the opposing second end.

46. The device according to claim 38, wherein the image capture component comprises a lens or a lens system.

47. The device according to claim 38, wherein the bone cutting element is selected from a reamer or a drilling element.

48. The device according to claim 47, wherein the drilling element forms an outer sheath to the elongate portion.

49. The device according to claim 38, wherein the elongate portion has a width or diameter of less than 10 mm.

50. The device according to claim 38, wherein the first end comprises a removable or retractable cover for the image capture component.

51. The device according to claim 38, wherein the device comprising a fixation component for securing the device in position in the bone tunnel with the image capture component in operable relation to the target area.

52. A method of inserting a scope into a joint of a subject in order to obtain an image of a target area in the joint, the method comprising forming a bone tunnel using the bone cutting element of the device of claim 38, and inserting the scope into the lumen of the device.

53. The method of claim 52, comprising securing the scope within the device.

54. A device for use in a method of obtaining an image of a target area in a joint of a subject, the device comprising:
an elongate portion having a first end and an opposing second end;
a bone cutting element at the first end for creating a bone tunnel;
a lumen surrounded by the bone cutting element at the first end, the lumen extending from the first end to the opposing second end;
a scope positioned in the lumen, wherein the scope comprises an image capture component at the first end; and
wherein the device comprises a securing part for reversibly securing a position of the device in the bone tunnel.
